(19) United States
(12) Patent Application Publication
(10) Pub. No.: US 2008/0194707 A1
(43) Pub. Date: Aug. 14, 2008

(54) Medical Devices

(76) Inventor: William Potter, Cambridgeshire (GB)

Correspondence Address: MCDERMOTT, WILL & EMERY LLP, 227 WEST MONROE STREET, SUITE 4400, CHICAGO, IL, 60606-5096, US

(21) Appl. No.: 11/659,377

(22) PCT Filed: Aug. 4, 2005
(86) PCT No.: PCT/GB2005/003061

§ 371 Date: Nov. 15, 2007

(30) Foreign Application Priority Data
Aug. 4, 2004 (GB) 0417350.6

(51) Int. Cl. A01N 25/10; A01P 1/00; A61M 27/00; A61F 5/44; A61L 29/00

(52) U.S. Cl. 514/772.4; 604/544; 604/328

(57) ABSTRACT

According to the invention there is provided a medical device capable of releasing a medically active ingredient, said device including a blend of: (i) a carrier polymer or a blend of carrier polymers; (ii) a medically active ingredient; and, optionally, (iii) a water sensitive polymer for releasing said medically active ingredient in the presence of water and/or a pH sensitive polymer for releasing said medically active ingredient in the presence of a solution having a pH within a predetermined range; with the proviso that, if component (iii) is absent, the carrier polymer includes an ethylene vinyl alcohol copolymer.

This invention relates to medical devices, and in particular to the controlled release of active ingredients from medical devices to prevent or treat microbial infection.

Urinary drainage bags are worn for a period of time, usually one week, during which time they repeatedly fill and are emptied. A tap is fitted to the bags to facilitate emptying. One of the problems associated with urinary drainage bags is that there is a risk of the urine in the bag becoming infected. If this occurs the infection can ascend through the catheter and infect the patient.

There is also a significant risk associated with infection of e.g. bacteria developing within the lumen of urinary catheters. Such infections are usually accompanied by the formation of a biofilm. Biofilms are aggregations of microorganisms surrounded by an extracellular matrix of exopolysaccharide. Bacteria are sandwiched between this polysaccharide coat and the catheter lumenal wall and are effectively isolated and separated from the surrounding urethral environment. This protection within the biofilm can lead to complications in executing an effective therapy against the bacteria. A key feature of infected urine is that many of the organisms involved e.g. Proteus mirabilis, produce ureases which are enzymes that cause the rapid breakdown of urea with the liberation of ammonia. As a consequence of the release of ammonia, the pH of the urine rises, causing the precipitation of crystalline material derived from the calcium and magnesium salts present in the urine. This crystalline material combines with the biofilm produced by the organism to form a crystalline biofilm which not only provides an ideal substrate within which and on which the contaminating organisms can grow, but also can block the catheter with serious medical consequences.

Catheters dipped and coated in antimicrobial solutions have been produced to address the problem of infections developing with urethral catheterization. See, for example, EP 0065884; EP 0426486; and U.S. Pat. No. 4,999,210. However, such prophylactic use of antibiotics to control bacteria present within urinary catheters has proved unsatisfactory. This is due to the fact that bacteria, which are protected by the polysaccharide coat of the biofilm, are not exposed to effective concentrations of the antimicrobial compound, and can still grow and multiply within the lumen of the catheter. Furthermore, the use of antibiotics in the absence of infection is of debatable merit because of drug side effects and the possibility of producing resistant strains.

To overcome these disadvantages, medical devices such as urinary bags and catheters have been manufactured with a coating which comprises an antimicrobial compound which is released by the coating in a certain manner affecting either the location or the timing of the release. The so-called “controlled release” of an active by such coatings has particular advantages in the context of administering active ingredients. For example, the release rate of a drug can be predicted and designed for an extended duration which eliminates problems associated with patients neglecting to take required medication in specified dosages at specified times. Another advantage of controlled release is that the half life of an active ingredient can be preserved by trapping the active in a polymeric matrix—this increases the time in which the drug maintains its activity. Furthermore, the release of an active at the site of local infection means that the active ingredient does not have to be administered systemically, and thus the side effects that certain medications cause when administered orally or intravenously can be avoided.

There are several general types of controlled release systems which are known in the art. For example, drug release can be diffusion controlled, meaning that the diffusion of the active ingredient trapped within a polymer matrix is the rate-determining factor for the overall release rate. Systems exist which are based on erosion of the coating, wherein the polymer degrades over time and releases an active ingredient in an amount proportional to the gradual erosion. Another system is based on the swelling of a polymeric matrix, such as a hydrogel. Hydrogels are polymers that absorb and swell in an aqueous environment. The release of the agent is dependent on the volume increase of the gel upon swelling and is then diffusion controlled. Hydrogels are well known and can be used for either coating a medical device such as a urinary catheter, or they can be formed in the shape of a tube for use as a catheter.

Another system exploits the rise in pH associated with the liberation of ammonia (due to urease producing bacteria), wherein the coating reacts to the onset of infection (as signalled by the rise in pH), to release more antimicrobial compound. Such release can be made slow enough so that the drug remains at significant levels for a clinically useful period of time. In this way the antimicrobial compound is used more effectively, the risk of exposure of the patient to the compound is reduced since it is only released at the onset of infection, and the duration of protection provided by a given amount of the compound can be extended. U.S. Pat. No. 5,788,687 discloses the use of pH-sensitive controlled release method, wherein a biologically active agent is released from a hydrophobic, pH-sensitive polymer matrix. In one embodiment, the polymer matrix swells when the environment reaches pH 8.5, releasing the active agent, and in another embodiment, the active agent is released as the pH drops.

There are however, significant problems associated with medical devices which comprise conventional hydrogel materials and water soluble polymers in conjunction with an active ingredient. Firstly, these systems invariably release the active ingredient too quickly. Secondly, they are also difficult to fabricate into useful devices, generally requiring the use of solvents and crosslinking agents. Thirdly, the materials themselves are also expensive and add significantly to the cost of devices made from them. Consequently, manufacturers have tended to restrict the types of antimicrobial agents that are used in order to reduce costs. For example, urinary catheters are available in which silver is used as the active ingredient, but the effectiveness of these products is limited.

The present invention, in at least some of its embodiments, overcomes the disadvantages of known controlled release medical devices, and provides improved medical devices (e.g. urinary drainage bags or catheters) which comprise a blend of components which can be used to release eg an antimicrobial compound in a controlled manner, in order to maintain an inhibitory concentration of the compound and so prevent infection, or reduce the risk of infection occurring within the device, eg, bag or catheter. Further advantages are that blends of readily and cheaply available conventional plastics and active ingredients can be utilised in the medical devices provided by the invention, and conventional production techniques can be employed to manufacture the blends and the medical devices of the present invention.

According to a broad aspect of the present invention there is provided a medical device capable of releasing a medically active ingredient, said device including a blend of:
i) a carrier polymer or a blend of carrier polymers;
ii) a medically active ingredient;
   and, optionally, iii) a water sensitive polymer for releasing said medically active ingredient in the presence of water and/or a pH sensitive polymer for releasing said medically active ingredient in the presence of a solution having a pH within a predetermined range;
   with the proviso that, if component iii) is absent, the carrier polymer includes an ethylene vinyl alcohol copolymer.

The medically active ingredient may be a drug, for example a drug to control inflammation or, preferably, an antimicrobial agent.

According to a preferred aspect of the present invention, then, there is provided a medical device capable of controlling a microbial infection, said device including a blend of:
i) a carrier polymer or a blend of carrier polymers;
ii) an antimicrobial agent;
   and, optionally, iii) a water sensitive polymer for releasing said antimicrobial agent in the presence of water and/or a pH sensitive polymer for releasing said antimicrobial agent in the presence of a solution having a pH within a predetermined range;
with the proviso that, if component iii) is absent, the carrier polymer includes an ethylene vinyl alcohol copolymer.

For the avoidance of doubt, the term ‘polymer’ as used herein includes within its scope copolymers. The term ‘ethylene vinyl alcohol copolymer’ is understood to be equivalent to the terms ‘polyethylene vinyl alcohol copolymer’ and ‘poly(ethylene-co-vinyl alcohol)’. When reference is made below to an antimicrobial agent, it is understood that other medically active ingredients might be utilised in place of the antimicrobial agent in some embodiments of the invention.

Preferably, the medical device is a urinary catheter or urinary drainage bag. The blend can be utilised in a number of ways. Preferably, the blend is present as a coating or an insert.

A coating may be used on all surfaces of the device or only on surfaces which are prone to microbial infection, for example the lumen of a catheter, or the interior of a urinary drainage bag. An insert can be positioned at any advantageous location within the medical device, e.g. within the urinary drainage bag itself. The insert may release an active antimicrobial agent into e.g. the urinary drainage bag, thereby preventing, or controlling a microbial infection. The medical device may be entirely fabricated from the blend, or component parts may be fabricated from the blend.

In preferred embodiments, the blend comprises between 1 and 50%, preferably between 1 and 40%, more preferably between 1 and 30%, more preferably still between 10 and 30%, by weight of the water sensitive polymer.

Additionally, or alternatively, a pH sensitive polymer may be utilised. In such instances, the blend may comprise between 1 and 40%, preferably between 1 and 30%, most preferably between 10 and 20% by weight of the pH sensitive polymer. In this way, it is possible to achieve a pH sensitive release of the antimicrobial agent.

In embodiments in which both a water sensitive polymer and a pH sensitive polymer are utilised, it is preferred that the blend comprises, in combination, between 1 and 50%, more preferably between 1 and 40% by weight of water sensitive polymer and the pH sensitive polymer, more preferably still between 1 and 30, most preferably between 10 and 30%, by weight.

The carrier polymer may be selected from the group consisting of: polyethylene, polypropylene, polyvinyl chloride, polyurethane, a polyolefin, polymers of vinyl esters, and copolymers thereof. Examples of ethylene copolymers include ethylene vinyl alcohol copolymer and ethylene vinyl acetate copolymer. Blends of the aforementioned polymers and copolymers may also be used as the carrier polymer. Preferred blends are polyethylene/ethylene vinyl alcohol copolymer and ethylene vinyl acetate copolymer/ethylene vinyl alcohol copolymer.

The carrier polymer may be essentially hydrophobic in nature, although the invention is not limited in this regard.

The antimicrobial agent may be a quaternary ammonium compound, preferably alkyl dimethyl benzyl ammonium chloride (hence forth termed BZK, although this compound is also known in the art as BAC). Other antimicrobial agents can be employed, such as other cationic compounds, metals, chlorhexidine gluconate or chlorhexidine acetate.

Surprisingly it has been found that in the case of ethylene vinyl alcohol copolymers, good release characteristics of the active antimicrobial agent can be achieved without the requirement for a water sensitive polymer and/or a pH sensitive polymer. Thus, a blend of the present invention may consist of ethylene vinyl alcohol copolymer in combination with an antimicrobial agent, the active antimicrobial agent being directly released from the ethylene vinyl alcohol copolymer. The ethylene vinyl alcohol copolymer may or may not be blended with another carrier polymer. If the ethylene vinyl alcohol copolymer is blended with another carrier polymer, preferred examples are blends of polyethylene with the ethylene vinyl alcohol copolymer or an ethylene vinyl acetate copolymer with the ethylene vinyl alcohol co-polymer.

In embodiments of the present invention, the blend typically comprises between about 0.1 and 20%, preferably between 0.1% and 15%, more preferably between 0.1 and 10%, most preferably between 1 and 10%, by weight of the antimicrobial agent.

The antimicrobial agent is optionally present on or in a carrier material in order to facilitate processing. Preferably, the carrier material is a silica or a clay, such as a bentonite. The antimicrobial agent may be adsorbed onto the carrier material or pre-mixed with the carrier material.

The water sensitive polymer is generally hydrophilic. The antimicrobial agent may be contained within the water sensitive release polymeric matrix, and may be released when the water sensitive release polymer hydrates, for example by contact with urine. The water sensitive polymer may be a water soluble polymer, in which instance the antimicrobial agent is released when the water soluble polymer contacts an aqueous solution (such as urine), and is dissolved thereby. Suitable water sensitive polymers include polyethylene oxide (PEO), for example of molecular weight in the range 500,000 to 1,000,000 or, preferably, polyvinyl alcohol. An example of a suitable polyvinyl alcohol has a degree of hydrolysis of 87% to 89% and a weight average molecular weight of 85,000 to 124,000. Polyvinyl alcohol may be blended with polyethylene or an ethylene vinyl acetate copolymer as the carrier polymer. Hydrogel polymers might be employed as an alternative, in which instance the antimicrobial agent is released when the hydrogel contacts an aqueous solution, absorbs water and swells. Examples of hydrogels can be found in Dimitrov et al, Acta Pharm, 53 (2003) 25 and references therein.

The pH sensitive polymer enables pH sensitive release of the antimicrobial agent to be achieved. By varying the nature of the pH sensitive polymer, it is possible to control the release profile of the antimicrobial agent as a function of pH. Polymers which become hydrophilic at pHs above 7, and thereby swell, causing release of the antimicrobial agent, are very useful. This is because the onset of infection in urine generally causes the pH of the urine to rise. Thus, antimicrobial agent is released when needed, and conserved when not. In contrast to hydrogels, pH sensitive polymers of this type swell to only minimal extents at low pH. The pH sensitive polymer can be a polymer containing an acid functional group, preferably containing carboxylic acid groups. Polymers containing acrylic or methacrylic acid are particularly preferred. In this way, pH sensitive release can be achieved. By selecting the composition of the acid containing pH sensitive polymer, the pH at which release of the antimicrobial agent begins can be controlled. Suitable pH sensitive polymers include acrylic copolymers, preferably containing acrylic or methacrylic acid. Particularly suitable polymers include the Eudragit (RTM) copolymers, such as Eudragit L400 (Pharma Polymere, a division of Rohm GmbH, Darmstadt, Germany).

In embodiments in which both a water sensitive polymer and a pH sensitive polymer are utilised, it is possible to vary the ratio of the water sensitive polymer to the pH sensitive polymer in order to control the release profile of the antimicrobial agent. Thus, for example, a release profile can be designed to give a low level of continuous release at low pHs coupled with a higher rates of release if, for example, urine becomes infected and pH rises.

In embodiments which comprise a water sensitive polymer and/or a pH sensitive polymer, the blend may further comprise a compatibilising agent for improving the dispersion of the water sensitive polymer and/or the pH sensitive polymer within the carrier polymer. The compatibilising agent is provided in such embodiments to ensure that the water sensitive polymer and/or pH sensitive polymer is adequately dispersed and incorporated into the blend. Typically, the compatibilising agent is present in an amount up to 10% by weight of the blend, preferably up to 5%. Typically, the compatibilising agent is a copolymer containing segments that are compatible with the carrier polymer and segments that are compatible with the water sensitive polymer and/or the pH sensitive polymer. Block and graft copolymers may be utilised as well as certain random copolymers. Block and graft copolymers of polyethylene are preferred. An example of a compatibilising agent is a polyethylene/maleic anhydride graft copolymer (polyethylene-graft-maleic anhydride) or a salt thereof. Other useful compatibilising agents include block copolymers of polyethylene and poly(ethylene glycol), poly(ethylene-co-methacrylic acid) copolymers or salts thereof, especially a sodium salt thereof although the use of, for example, lithium and zinc salts is possible, and poly(ethylene-co-acrylic acid) or salts thereof. The use of the compatibilising agent is particularly preferred in order to disperse a water sensitive polymer. It has been found that pH sensitive polymers generally do not require a compatibilising agent, although the use of a compatibilising agent in conjunction with a pH sensitive polymer is within the scope of the invention.

The microbial infection may cause a change in the pH of the environment of the device, for example due to the liberation of ammonia during the urease mediated degradation of urine. The active antimicrobial agent may be released from the pH sensitive release polymer upon the change in the pH.

According to a further broad aspect of the present invention, there is provided a process for producing a blend for use in a medical device capable of releasing a medically active ingredient, the blend including:
i) one or more carrier polymers;
ii) a medically active ingredient;
   and, optionally, iii) a water sensitive polymer for releasing said medically active ingredient in the presence of water and/or a pH sensitive polymer for releasing said medically active ingredient in the presence of a solution having a pH within a predetermined range;
with the proviso that, if component iii) is absent, the carrier polymer includes an ethylene vinyl alcohol copolymer,
   said process comprising the step of mixing components i), ii) and, optionally, iii) together to form said blend.

According to a further preferred aspect of the present invention, there is provided a process for producing a blend for use in a medical device for controlling a microbial infection, the blend including:
i) one or more carrier polymers;
ii) an antimicrobial agent;
   and, optionally, iii) a water sensitive polymer for releasing said antimicrobial agent in the presence of water and/or a pH sensitive polymer for releasing said antimicrobial agent in the presence of a solution having a pH within a predetermined range;
with the proviso that, if component iii) is absent, the carrier polymer includes an ethylene vinyl alcohol copolymer,
said process comprising the step of mixing components i), ii) and, optionally, iii) together to form said blend.

The process may further comprise the step of extruding the blend. Indeed, it is an advantage of the present invention that the blends provided thereby can be easily extruded using standard production techniques to form films, inserts or coatings which are suitable for use with, or in, such medical devices. For the avoidance of doubt, the process of co-extrusion is, in the context of the present invention, understood to represent an example of an extrusion process. The process may further comprise the step of coating a medical device with a film of the blend, and/or may further comprise the step of forming an insert for a medical device from the blend.

### EXAMPLES

The following examples detail the antimicrobial activity of blends having direct release and controlled release characteristics. In the examples the given percentages represent percentage by weight of a component with respect to the weight of the composition including that component.

### Direct Release Formulations

In all cases blends were manufactured as films using extrusion. The films were evaluated for antimicrobial activity using one the following procedures:

### Zone of Inhibition Test

Two 0.5 inch squares from each extruded sample were immersed in nutrient agar seeded with E. coli, P. fragi, B. epidermidis or Br. thermosphacta, and Tryptone Soya Agar seeded with L. innocua. All seeded plates were incubated at 30° C. for 48 h and the sizes of the zones of inhibition around the sample were recorded.

### Broth Extraction Test

Extruded film samples (5 g) were each extracted with 100 ml Iso-sensitest broth at 37° C. The Iso-sensitest broth was replenished at 24 hour intervals. Samples of the extracts were assessed for BZK content. Wells were cut into nutrient agar plates seeded with 1 ml of E. coli at 1×10⁵ cfu/ml. Doubling dilutions of the extracts (1, ½, ¼, ⅛) were prepared and 200 μl added to the appropriate well. Plates were incubated at 30° C. for 24 h to determine the limits of dilution at which zones of inhibition formed. The concentration of BZK was estimated from the reciprocal of the dilution at which inhibition still occurred.

### BZK Release Test

Daily release rates for BZK were measured by extracting 6 g of each film daily with a fresh aliquot of 200 ml of water. Samples of extract from days 1 and 4 analysed for BZK content by HPC. The amount of BZK released in the 24 hour period is expressed as a percentage of the initial BZK content in the film.

### Example 1

### Low Density Polyethylene Blends

Blends of low density polyethylene (LDPE) with various components were extruded using a laboratory scale extruder to form thin films. Samples cut from each film were subjected to the zone of inhibition test, or BZK release test, as described above. The results of the zone of inhibition test (measured in mm) are displayed in Table 1.

TABLE 1
Zone of inhibition test for low density polyethylene blends

| Composition | E. coli | B. epidermidis | L. innocua | Br. thermosphacta | P. fragi |
|---|---|---|---|---|---|
| 80% LDPE, 20% PEO | 0 | 0 | 0 | 0 | 0 |

This result shows that blends based on LDPE without BZK are not active. This is unsurprising since the blend does not contain any active antimicrobial agent.

TABLE 2
BZK Release rates for low density polyethylene blends

| Composition (%) | | | | | | BZK | |
|---|---|---|---|---|---|---|---|
| | | | | PE-MA | | Release (%) | |
| LDPE | EVA | PVOH | EVOH | Graft | BZK | Day 1 | Day 4 |
| 81 | | 9 | | 5 | 5 | 15.80 | 5.87 |
| 63 | | 27 | | 5 | 5 | 11.80 | 5.33 |

Table 2 shows release rates for various blends of LDPE with BZK which can include polyvinyl alcohol (PVOH), and a polyethylene-maleic acid graft copolymer (PE-MA Graft). Riblene FF 24 film grade LDPE, and 87 to 89% hydrolysed, 85000 to 124000 Mw PVOH (Aldrich) were used. PE-MA Graft having a melt index of 1.50 g/10 min was also obtained from Aldrich.

These results demonstrate that a blend containing LDPE, PVOH, a compatibiliser (PE-MA graft) and 5% BZK can release BZK for periods of four days (and possibly longer). Separate studies have shown that the amounts of BZK released are sufficiently high to be effective against bacteria such as E. coli. The films are of good flexibility.

### Example 2

### Ethylene Vinyl Alcohol Copolymer Blends

Mixtures of ethylene vinyl alcohol copolymers (EVOH) with various components were extruded using a laboratory scale extruder to form thin sheets. EVOH products Eval L101B and Eval F101B, obtained from Eval Europe, a division of Kuraray, were used. Samples cut from each film were subjected to the zone of inhibition test, and broth extraction test, as described above. The results of the zone of inhibition test (measured in mm) are displayed in Table 3.

TABLE 3
Zone of inhibition test for ethylene vinyl alcohol co-polymers blends

| Composition | E. coli | B. epidermidis | L. innocua | Br. thermosphacta | P. fragi |
|---|---|---|---|---|---|
| 100% EVOH | 0 | 0 | 0 | 0 | 0 |
| 90% EVOH, 10% PEO | 0 | 0 | 0 | 0 | 0 |
| 80% EVOH, 20% PEO | 0 | 0 | 0 | 0 | 0 |
| 60% EVOH, 20% PEO, 20% BZK | 2 | 0 | 6 | 0 | 0 |
| 70% EVOH, 10% PEO, 20% BZK | 2 | 0 | 2 | 7 | 2 |

The estimated concentration of BZK extracted for each 24 hour interval expressed in arbitrary units as the reciprocal of the dilution factor are shown in the following table:

TABLE 4
Broth extraction test for ethylene vinyl alcohol co-polymer blends

| Composition | 24 h | 48 h | 72 h | 96 h |
|---|---|---|---|---|
| 100% EVOH | 8 | 0 | 0 | 0 |
| 90% EVOH, 10% PEO | 2 | 0 | 0 | 0 |
| 80% EVOH, 20% PEO | 0 | 0 | 0 | 0 |
| 60% EVOH, 20% PEO, 20% BZK | >8 | 8 | 8 | 8 |
| 70% EVOH, 10% PEO, 20% BZK | >8 | 8 | 8 | 8 |

Results using ethylene vinyl alcohol co-polymer blends show that blends containing no BZK exhibited zero activity in terms of zones of inhibition against each of the bacterial species tested. This finding is consistent with the results obtained in the broth extraction test, where the extracted samples after 24 hours failed to exhibit any antimicrobial activity. The blend containing 60% EVOH, 20% PEO, 20% BZK exhibited some antimicrobial activity against E. coli and L. innocua, but no antimicrobial activity against the other organisms tested. Importantly, the Broth extraction test results listed in Table 4, reveal that the activity against E. coli was sustained for 4 days incubation, and that the amount of BZK released over this time period was consistently high. The blend containing 70% EVOH, 10% PEO, 20% BZK exhibited a greater spectrum of activity against the organisms tested (Table 3), and consistent with the blend containing 60% EVOH, 20% PEO, 20% BZK, the antimicrobial activity against E. Coli was sustained for 4 days incubation, and the amount of BZK released over this time period was consistently high (Table 4).

Films having improved flexibility were made using Eval ES104. Films were made by extruding a mixture of 95% Eval ES 104 and 5% BZK. The BZK release profile for this film was excellent with 11.7% of the BZK being released on day 1 and 9.9% being released on day 4.

### Example 3

### Low Density Polyethylene Blends and Ethylene Vinyl Alcohol Copolymer Blends

In further experiments, blends based on both LDPE and EVOH were extruded. In contrast to the experiments described for each of these blends above, the base polymers in combination with BZK were tested with and without a water sensitive release polymer. The results obtained in the zone of inhibition test (measured in mm) around samples cut from the films when challenged with E. coli are displayed in Table 5.

TABLE 5
Zone of inhibition test for low density polyethylene blends and
ethylene vinyl alcohol co-polymer blends

| | Zone of Inhibition |
|---|---|
| Composition | (mm) |
| 100% LDPE | 0 |
| 90% LDPE, 10% BZK | 0 |
| 90% EVOH (Eval L101B), 10% BZK | 30.5 |
| 80% EVOH (Eval L101B), 10% PEO, 10% BZK | 32 |
| 80% EVOH (Eval F101A), 10% PEO, 10% BZK | 34.5 |
| 60% EVOH (Eval F101A), 20% PEO, 20% BZK | 33.5 |

Blends consisting of ethylene vinyl alcohol copolymer (as a base polymer), polyethylene oxide (as a water sensitive release polymer), and BZK, exhibit antimicrobial activity consistent with the results shown in Table 3. Surprisingly, this experiment revealed that a blend of ethylene vinyl alcohol co-polymer (as a base polymer) in combination with BZK only, and in the absence of any water sensitive release polymer, exhibited antimicrobial activity against E. coli. The release of BZK by the base polymer was as effective as those blends containing 10% or 20% PEO as a water sensitive release polymer. Seemingly, the release of BZK by a base polymer in the absence of a water sensitive release polymer was confined to blends consisting of ethylene vinyl alcohol co-polymer, and was not apparent in a blend consisting of LDPE and BZK only.

Further studies have shown that blends of ethylene vinyl alcohol copolymers with other polymers such as polyethylene and ethylene vinyl acetate copolymers (EVA) containing BZK also have excellent release profiles. The EVA utilised was Evathane ex ATO. These films also have the advantage of being flexible. Examples of such films are given in Table 6 below:

TABLE 6
Release profiles for EVOH blends with polyethylene
and ethylene vinyl acetate copolymers

| Composition (%) | | | | | | | BZK Release (%) | |
|---|---|---|---|---|---|---|---|---|
| LDPE | EVA | PVOH | EVOH | PE-MA Graft | BZK | Eval Grade | Day 1 | Day 4 |
| 72 | | | 18 | 5 | 5 | F101 | 2.93 | 2.20 |
| | 63 | | 27 | 5 | 5 | F101 | 9.53 | 5.40 |

### Example 4

### pH Sensitive Release Formulations

A blend of Eudragit L400 and BZK was prepared by dissolving 67 parts of Eudragit L400 and 33 parts of BZK in isopropanol, drying off the solvent and grinding the resulting mass to a powder. Blends of LDPE and PEO containing the powder were extruded and zones of inhibition were measured at different pH values by using appropriate agar formulations. The zones of inhibition around samples cut from the extruded films when challenged with E. coli are shown in Table 6.

TABLE 6
Zone of inhibition test for LDPE pH sensitive release blends

| | | Zone of Inhibition |
|---|---|---|
| Conditions | Film | (mm) |
| pH = 6 | 85% LDPE 15% Eudragit/BZK | 4 |
| pH = 7 | 85% LDPE 15% Eudragit/BZK | 17 |
| pH = 8 | 85% LDPE 15% Eudragit/BZK | 23 |
| pH = 6 | 70% LDPE 15% PEO 15% Eudragit/BZK | 5 |
| pH = 7 | 70% LDPE 15% PEO 15% Eudragit/BZK | 19.5 |
| pH = 8 | 70% LDPE 15% PEO 15% Eudragit/BZK | 45 |

The results clearly show that the release of BZK, as determined by the zone of inhibition, increase as the pH rises. Furthermore, the presence of PEO as a direct release water sensitive release polymer contributes significantly to the release of BZK at pH 8.0.

### Example 5

### Solvent Cast pH Sensitive Release Formulations

The following experiments show that pH sensitive blends (cast from solvents) also release active antimicrobial agent in a pH dependent manner.

Test samples were prepared by adding 0.47 g tri-ethyl citrate to 60 g isopropanol and then dissolving 18 g Eudragit L100 in stages with rapid stirring. 3 g BZK was added when all the Eudragit had dissolved. Blocks were cast and the isopropanol was removed by evaporation over 96 hours.

Samples removed from the cast blocks were added to 100 ml Iso-sensitest broth at pH 6.0 or 8.0 and incubated at 37° C. for 24 hour intervals. The broth was replenished every 24 hours with either pH 6.0 or pH 8.0 broth such that the samples were alternately exposed to high and low pH. As before the BZK content of the extracts was assessed by determining the limiting dilution which inhibition of E. coli still occurred.

The concentrations of BZK expressed in arbitrary units are shown in Table 7.

TABLE 7
Broth extraction test for solvent cast pH sensitive release formulations

| | 24 h pH 6 | 48 h pH 8 | 72 h pH 6 | 96 h pH 8 |
|---|---|---|---|---|
| Sample 1 | 1 | 4 | 0 | 4 |
| Sample 2 | 0 | 4 | 0 | 8 |

The results shown in Table 7 reveal that BZK was not released from the blends at pH 6, but was released from the blends at pH 8. This release decreased at 72 h when the pH was reduced to 6, but resumed when the pH was restored to 8, confirming that BZK was released in a pH dependent manner. It should be noted that very little BZK was extracted in the first 24 hours suggesting that some time is required for the samples to hydrate initially.

The examples demonstrate that active antimicrobial agents can be released in a sustained and continual manner whereby the active antimicrobial agent remains at an effective concentration for a clinically useful period of time (i.e four days or more). This release is a significant improvement over prior art direct release formulations, which tend to release their active antimicrobial agents too quickly, and in a non-sustainable manner. Advantageously, the formulations according to the present invention can be manufactured cheaply by standard production methodologies, using cheap and readily available plastic materials. It has also been shown that active antimicrobial agents can be released from a blend consisting only of a carrier polymer and active antimicrobial agent, without the requirement for a release polymer. The direct release formulations according to the present invention therefore offer considerable advantages over known polymeric blends which have been previously used in medical devices.

Active antimicrobial agents can be released in a pH dependent manner, using pH sensitive release formulations according to the present invention. In this way, the active antimicrobial agent is released at an effective concentration for a clinically useful period of time (i.e four days or more). Also, it is demonstrated that a blend comprising a direct release water sensitive polymer in combination with a pH sensitive release polymer provides release of an active antimicrobial agent over a clinically useful period of time, whereby the active antimicrobial agent is released in large and sustained concentrations. This controlled release, or indeed controlled release combined with direct release represents a significant improvement over prior art controlled release formulations, which tend to release their active antimicrobial agents either too quickly, and/or in a non-sustainable manner. Similar to direct release formulations, pH sensitive release formulations according to the present invention can be manufactured cheaply by standard production methodologies, using cheap and readily available plastic materials. The pH sensitive controlled formulations according to the present invention therefore offer considerable advantages over known polymeric blends which have been previously used in medical devices.

We claim:

1. A medical device capable of releasing a medically active ingredient, said device including a blend of:
i) a carrier polymer or a blend of carrier polymers;
ii) a medically active ingredient; and, optionally,
iii) a water sensitive polymer for releasing said medically active ingredient in the presence of water and/or a pH sensitive polymer for releasing said medically active ingredient in the presence of a solution having a pH within a predetermined range;
with the proviso that, if component iii) is absent, the carrier polymer includes an ethylene vinyl alcohol copolymer.

2. A medical device according to claim 1 capable of controlling a microbial infection, said device including a blend of:
i) a carrier polymer or a blend of carrier polymers
ii) an antimicrobial agent; and, optionally,
iii) a water sensitive polymer for releasing said antimicrobial agent in the presence of water and/or a pH sensitive polymer for releasing said antimicrobial agent in the presence of a solution having a pH within a predetermined range;
with the proviso that, if component iii) is absent, the carrier polymer includes an ethylene vinyl alcohol copolymer.

3. A medical device according to claim 1 in which the blend is present as a coating or an insert.

4. A medical device according to claim 1 in the form of an urinary catheter or urinary drainage bag.

5. A medical device according to claim 1 in which the blend includes between 0.1 and 20%, preferably between 0.1 and 15%, more preferably between 0.1 and 10%, most preferably between 1 and 10%, by weight of the medically active ingredient.

6. A medical device according to claim 1 any previous claim in which the blend includes between 1 and 50%, preferably between 1 and 40%, more preferably between 1 and 30%, most preferably between 10 and 30%, by weight of the water sensitive polymer.

7. A medical device according to claim 1 in which the blend includes between 1 and 40%, preferably between 1 and 30%, most preferably between 10 and 20%, by weight of the pH sensitive polymer.

8. A medical device according to claim 1 in which the blend includes, in combination, between 1 and 50%, preferably between 1 and 40% by weight of the water sensitive polymer and the pH sensitive polymer, more preferably between 1 and 30%, most preferably between 10 and 30%, by weight.

9. A medical device according to claim 1 in which said carrier polymer is selected from the group consisting of: polyethylene, polypropylene, polyvinyl chloride, polyurethane, a polyolefin, polymers of vinyl esters, and copolymers thereof.

10. A medical device according to claim 1 in which a carrier polymer is an ethylene vinyl alcohol copolymer or an ethylene vinyl acetate copolymer.

11. A medical device according to claim 10 including a blend of polyethylene and ethylene vinyl alcohol copolymer carrier polymers or a blend of polyethylene and ethylene vinyl acetate copolymer carrier polymers.

12. A medical device according to claim 2 in which said antimicrobial agent is a quaternary ammonium compound, preferably benztrimethylammonium chloride.

13. A medical device according to claim 1 in which the medically active ingredient is present on or in a carrier material.

14. A medical device according to claim 13 in which the carrier material is a silica or a clay.

15. A medical device according to claim 13 in which the medically active ingredient is adsorbed onto the carrier material.

16. A medical device according to claim 1 in which the water sensitive polymer is a water soluble polymer.

17. A medical device according to claim 16 in which the water sensitive polymer is polyvinyl alcohol or polyethylene oxide.

18. A medical device according to claim 17 in which the water sensitive polymer is polyvinyl alcohol, and the carrier polymer is polyethylene or ethylene vinyl acetate copolymer.

19. A medical device according to claim 1 in which the pH sensitive polymer is a polymer containing an acid functional group, preferably a carboxylic acid group, most preferably containing acrylic or methacrylic acid.

20. A medical device according to claim 19 in which the pH sensitive polymer is a methacrylic copolymer.

21. A medical device according to claim 1 including component iii) and further including a compatibilising agent for improving the dispersion of the water sensitive polymer and/or the pH sensitive polymer within the carrier polymer.

22. A medical device according to claim 21 in which the compatibilising agent is present in an amount up to 10% by weight of the blend, preferably up to 5%.

23. A medical device according to claim 21 in which the compatibilising agent is a copolymer, preferably block or graft copolymer.

24. A medical device according to claim 23 in which said compatibilising agent is a polyethylene/maleic anhydride graft copolymer or a salt thereof.

25. A process for producing a blend for use in a medical device capable of releasing a medically active ingredient, the blend including:
i) one or more carrier polymers;
ii) a medically active ingredient; and, optionally,
iii) a water sensitive polymer for releasing said medically active ingredient in the presence of water and/or a pH sensitive polymer for releasing said medically active ingredient in the presence of a solution having a pH within a predetermined range;
with the proviso that, if component iii) is absent, the carrier polymer includes an ethylene vinyl alcohol copolymer,
said process comprising the step of mixing components i), ii) and, optionally, iii) together to form said blend.

26. A process according to claim 25 for producing a blend for use in a medical device for controlling a microbial infection, the blend including:
i) one or more carrier polymers;
ii) an antimicrobial agent; and, optionally,
iii) a water sensitive polymer for releasing said antimicrobial agent in the presence of water and/or a pH sensitive polymer for releasing said antimicrobial agent in the presence of a solution having a pH within a predetermined range;
with the proviso that, if component iii) is absent, the carrier polymer includes an ethylene vinyl alcohol copolymer,
said process comprising the step of mixing components, i, ii) and, optionally, iii) together to form said blend.

27. A process according to claim 25 further including the step of extruding the blend.

28. A process according to claim 25 further including the step of coating a medical device with a film of the blend.

29. A process according to claim 25 further including the step of forming an insert for a medical device from the blend.
